(19) United States
(12) Patent Application Publication
(10) Pub. No.: US 2010/0174278 A1
(43) Pub. Date: Jul. 8, 2010

(54) METHODS OF NASOPHARYNGEAL COOLING FOR AUGMENTING CORONARY PERFUSION PRESSURE

(76) Inventors: DENISE BARBUT, New York, NY (US); Wanchun Tang, Rancho Mirage, CA (US); Allen Rozenberg, San Diego, CA (US)

Correspondence Address: O''Melveny & Myers LLP;IP&T Calendar Department LA-13-A7, 400 South Hope Street, Los Angeles, CA, 90071-2899, US

(21) Appl. No.: 12/614,274

(22) Filed: Nov. 6, 2009

Related U.S. Application Data
(60) Provisional application No. 61/112,622, filed on Nov. 7, 2008.

(51) Int. Cl. A61B 18/02

(52) U.S. Cl. 606/21

(57) ABSTRACT

A method for improving the success of resuscitation efforts following cardiac arrest is provided. Return of spontaneous circulation (ROSC) rates following cardiac arrest is directly related to the coronary perfusion pressure during cardiopulmonary resuscitation (CPR). Selective cooling of the nasal cavity, nasopharynx, oral cavity, oropharynx, retrotonsillar space, mouth, neck face, and/or throat of a patient suffering from cardiac arrest, significantly increases the coronary perfusion pressure which improves ROSC rates. Cooling may be initiated before or during resuscitation efforts including chest compressions, defibrillation and/or administering a vasoconstrictor.

### RELATED APPLICATIONS

This application claims the benefit of U.S. Application Ser. No. 61/112,622, filed Nov. 7, 2008, which is hereby expressly incorporated by reference in its entirety.

### FIELD OF THE INVENTION

The field of the invention generally relates to methods of increasing coronary perfusion pressure (CPP) in patients suffering from cardiac arrest to improve the success of return of spontaneous circulation (ROSC). More particularly, the field of the invention relates to methods of cooling an anatomic location including the nasal cavity, nasopharynx, oral cavity, oropharynx, retrotonsillar space, mouth, neck, face or throat before or during resuscitation from cardiac arrest to increase CPP.

### BACKGROUND OF THE INVENTION

A cardiac arrest is the cessation of normal circulation of the blood due to failure of the ventricles of the heart to contract effectively resulting in the cessation of blood delivery to the whole body. As a consequence cells of the whole body suffer injury that result from oxygen starvation. Lack of oxygen supply to the brain causes victims to immediately lose consciousness and stop breathing. Cardiac arrest is different from a heart attack (myocardial infarction). In a cardiac arrest the heart suddenly stops beating. During a heart attack, only a part of the heart ceases to work properly; the rest of the heart muscle continues to work promoting blood flow at a somewhat diminished level. However, heart attacks can sometimes lead to cardiac arrest in which the heart as whole stops beating and ceases to promote blood flow into the systemic circulation.

Cardiac arrest is often precipitated by ventricular fibrillation. Ventricular fibrillation most often occurs associated with underlying coronary artery disease in which case ventricular fibrillation may be the initial manifestation of a heart attack. However, ventricular fibrillation may also occur as a result of electrical abnormalities of the heart muscle, trauma to the heart, diseases that affect the heart muscle such cardiomyopathies, congenital or acquired abnormalities that regulate the way in which the electrical impulse of the heart is initiated and propagated; administration of drugs that can alter such ion channels, abnormalities in the chemical composition of the blood, or abnormalities in the valves of the heart.

Cardiac arrest can also occur without ventricular fibrillation, in which case the heart stops beating because of asystole in which there is no electrical impulses originating from the heart, or because of pulseless electrical activity in which electrical impulses originating from the heart are not effective to promote normal contraction of the heart muscle. Cardiac arrest caused by asystole or pulseless electrical activity is typically associated with conditions leading to severe curtailment of the amount of oxygen delivered to the heart muscle, which can occur as a result of respiratory failure or severe loss of circulating blood volume. Cardiac arrest caused by asystole or pulseless electrical activity can also occur associated with existing cardiac disease, especially when severe heart failure has developed.

Resuscitation treatments for patients suffering from cardiac arrest generally include clearing and opening the patient's airway, providing rescue breathing and applying manual chest compressions to provide blood flow to the victim's heart, brain and other vital organs. When all three treatments are combined, the term cardiopulmonary resuscitation (CPR) is used. Advanced cardiac support (ACS) may also be provided in the form of drugs, defibrillation, and other techniques.

The main factor determining the success of the resuscitation effort following cardiac arrest is coronary perfusion pressure (CPP) during cardiopulmonary resuscitation (CPR). The better the coronaries are perfused, the better the myocardium is irrigated and the easier it is to restore a heartbeat with return of spontaneous circulation (ROSC). Chest compressions will increase coronary perfusion pressure, however chest compressions alone typically do not provide a sufficient increase in CPP for successful resuscitation

When coronary perfusion pressure (CPP) is below 12-14 mm Hg during chest compression, the chances of establishing ROSC are very low. CPP is typically measured as the difference between the aortic pressure and the right atrial pressure. Epinephrine is often used during resuscitation in an attempt to increase aortic pressure and hence CPP, although its benefit is unclear. Any maneuver which increases CPP is invaluable in this setting. Therefore, there is a need for ways of improving or enhancing the efficacy of CPR measures or treatments by increasing the coronary perfusion pressure.

### SUMMARY OF THE INVENTION

The present invention provides for methods of increasing coronary perfusion pressure in patients suffering from cardiac arrest to improve the success of return of spontaneous circulation (ROSC).

In general, we have determined that cooling the nasal cavity, nasopharynx, oral cavity, oropharynx, retrotonsillar space, mouth, neck, face or throat before or during resuscitation from cardiac arrest increases CPP by augmenting regional sympathetic activity, for example by increasing the sympathetic tone at the aortic root/ascending aorta. This causes increased aortic pressure and therefore increases the coronary perfusion pressure. The anatomy of the brain is such that cooling the nasopharynx and/or oropharyx cools one or more of the cardiac or autonomic control centers, the hypothelamopituitary axis, the midbrain, the pons, the medulla, the autonomic fibers around the brain stem, the cervical sympathetic chain and the thoracic sympathetic chain. In certain cases, cooling in the nasopharynx and/or oropharynx anesthetizes nerve endings and changes the firing pattern in the cold sensing neurons in the nose, mouth or brain. For example, the typical hemodynamic response to cold exposure is vital organ presentation thus when the hypothalamus receives a message from the oral or nasal cavity nerve endings regarding cooling, the hypothalamus responds by sending a message to increase the CPP.

In one aspect of the invention, a method for increasing coronary perfusion pressure to improve success of return of spontaneous circulation (ROSC) after cardiac arrest is described. A patient suffering from cardiac arrest is selected. An anatomic location selected from the group consisting of, nasopharynx, nasal cavity, oropharynx, retrotonsillar space, mouth, neck and throat is cooled to increase the coronary perfusion pressure and chest compressions, defibrillation and/or a vasoconstrictor are administered to the patient. In some embodiments, the anatomic location may be cooled for less than 20 minutes, for between about 10-20 minutes, alternatively no more than 5 minutes. The cooling may be initiated before or during resuscitation. The cooling may increase the coronary perfusion pressure within 1-5 minutes, alternatively within 1-3 minutes, alternatively within 1-30 seconds, alternatively within 1-5 seconds. The coronary perfusion pressure may remain elevated for more than 3 minutes, alternatively more than 5 minutes, alternatively for at least 10 minutes.

In another aspect of this invention, the anatomic location may be cooled by circulating a cold liquid through the anatomic location. The cold liquid may be selected from chilled saline, water, water containing a nanotech particle, a perfluorocarbon, a hydrocarbon or an ice slurry. Alternatively, the anatomic location may be cooled by circulating a cold gas. The cold gas may be selected oxygen or compressed air. In an alternative embodiment, a cold liquid and gas mixture maybe used to cool the anatomic location.

The anatomic location may be cooled for less than 20 minutes, alternatively between 10-20 minutes, alternatively no more than 5 minutes. In some embodiments, the cold liquid may be circulated continuously, for example at a flow rate of between 1 ml/min-500 ml/min. In alternative embodiments, the cold liquid may be circulated intermittently. The intermittent circulation may have a duty cycle of between 5-80%. In some embodiments, a pulsatile spray may be used to deliver the cold liquid intermittently.

In another aspect of the invention, the cooling the anatomic location increases the coronary perfusion pressure without reducing the patient's brain temperature. For example, cooling the anatomic location may increase the coronary perfusion pressure while maintaining the patient's baseline brain temperature at 37° C., alternatively at between about 35-40° C. In some embodiments, cooling the anatomic location increases the coronary perfusion pressure significantly with out any measurable brain cooling. In alternative embodiments, cooling the anatomic location increases the coronary perfusion pressure significantly before any measurable brain cooling. For example, in one aspect of the invention, cooling the anatomic location increases the coronary perfusion pressure by at least 25%, alternatively at least 30%, alternatively at least 40%, alternatively at least 50%, within the first minute, alternatively within 2 minutes, alternatively within 3 minutes, alternatively within 4 minutes, alternatively within 5 minutes, alternatively within 10 minutes, alternatively within 15 minutes without reducing the brain temperature.

In one aspect of the invention, cooling the anatomic location increases the CPP and results in a return of spontaneous circulation (ROSC) without any measurable brain cooling. In some embodiments, cooling the anatomic location to increase CPP may also eventually result in the patient's brain temperature being reduced by between about 0.01-5° C.

In some embodiments cooling one of the anatomic locations may also cool one or more of the cardiac control centers in the brain. Cooling on one or more of the cardiac control centers may change the firing pattern in the cold sensing neurons in the mouth, in the nose, in the underside frontal lobe or preoptic region of the brain or in the brain stem.

In another aspect of this invention, a method for increasing the coronary perfusion pressure by cooling the nasal cavity is described. The temperature in the nasal cavity may be reduced by 0.01-20° C. in order to augment regional sympathetic activity and thereby increase the CPP. In some embodiments, cooling the nasal cavity increases the coronary perfusion pressure significantly without any measurable brain cooling. In alternative embodiments, cooling the nasal cavity increases the coronary perfusion pressure significantly before any measurable brain cooling. For example, in one aspect of the invention, the reduction in nasal cavity temperature and increase in CPP occurs without any measurable cooling of the brain, i.e. the baseline brain temperature of the patient is maintained between about 35-40° C., preferably 37° C. In an alternative embodiment, cooling the nasal cavity may also eventually result in the patient's brain temperature being reduced by between about 0.01-5° C. In another aspect of the invention, cooling the nasopharynx may also result in cooling one or more of the hypothelamopituitary axis, the midbrain, the pons, the medulla, the autonomic fibers around the brain stem, the cervical sympathetic chain and or/the thoracic sympathetic chain.

In another aspect of this invention, the nasal cavity may be cooled by circulating a cold liquid through the nasal cavity. The cold liquid may be selected from chilled saline, water, water containing a nanotech particle, a perfluorocarbon, a hydrocarbon or an ice slurry. In some embodiments, the cold liquid may be circulated continuously, for example at a flow rate of between 1 ml/min-500 ml/min. In alternative embodiments, the cold liquid may be circulated intermittently. The intermittent circulation may have a duty cycle of between 5-80%. In some embodiments, a pulsatile spray may be used to deliver the cold liquid intermittently. For example, in some embodiments, a nasal catheter, such as a Rhinochill™ device (BeneChill, San Diego, Calif.) or similar nasal catheter, may be used to deliver a cold liquid, or alternatively a cold liquid in combination with a gas, continuously or intermittently.

In another aspect of this invention, the nasal cavity and/or nasopharynx may be cooled by circulating a cold gas through the nasal cavity. The cold gas may be selected oxygen or compressed air. In an alternative embodiment, a cold liquid and gas mixture maybe used to cool the nasal cavity.

In another aspect of this invention, a method for increasing the coronary perfusion pressure by cooling the oropharynx and/or oral cavity is described. In some embodiments, the temperature in the mouth may be reduced to between about −10 and 30° C., alternatively to between about −2 and 10° C. in order to augment regional sympathetic activity and thereby increase the CPP. In some embodiments, the reduction in oral cavity temperature and increase in CPP may occur without any measurable cooling of the brain, i.e. the baseline brain temperature of the patient is maintained between about 35-40° C., preferably 37° C. In alternative embodiments, cooling the oral cavity may also eventually result in the patient's brain temperature being reduced by between about 0.01-5° C.

The oropharynx and/or oral cavity may be cooled by circulating a cold liquid through the oral cavity. The cold liquid may be selected from chilled saline, water, water containing a nanotech particle, a perfluorocarbon, a hydrocarbon or an ice slurry. In some embodiments, the cold liquid may be circulated continuously, for example at a flow rate of between 1 ml/min-500 ml/min. A transoral cooling assembly, such as a modified laryngeal mask, can be used to circulate the cold liquid. In alternative embodiments, the cold liquid may be circulated intermittently. The intermittent circulation may have a duty cycle of between 5-80%. In some embodiments, a pulsatile spray may be used to deliver the cold liquid intermittently. For example, in some embodiments, a trans-nasal or trans-oral catheter may be used to deliver a cold liquid, or alternatively a cold liquid in combination with a gas, continuously or intermittently to the oropharynx and/or oral cavity.

### BRIEF DESCRIPTION OF THE DRAWINGS

FIG. 1A is a schematic diagram showing the study design for illustrating the increase in coronary perfusion pressure (CPP) from nasal cooling in a Pulseless Electrical Activity (PEA) model of cardiac arrest in pigs.

FIG. 1B is a table illustrating the increase in CPP during CPR for a PEA model of cardiac arrest in pigs.

FIG. 1C illustrates the improved ROSC resulting from the increased CPP illustrated in FIG. 1A.

FIG. 2A is a schematic diagram showing the study design for illustrating the increase in coronary perfusion pressure (CPP) from nasal cooling in a Ventricular Fibrillation (VF) model of cardiac arrest in pigs.

FIG. 2B is a table illustrating the increase in CPP during CPR in a VF model of cardiac arrest in pigs.

FIG. 3A is a schematic diagram showing an alternative study design for illustrating the increase in coronary perfusion pressure (CPP) from nasal cooling in a prolonged ventricular fibrillation (VF) model of cardiac arrest in pigs.

FIG. 3B is a table illustrating the increase in CPP with nasopharyngeal and/or oral cooling in a prolonged VF model of cardiac arrest in pigs.

FIG. 3C is a table illustrating the increase in CPP with nasopharyngeal and/or oral cooling in a VF model of cardiac arrest in pigs dependant on the cooling dose rate.

FIG. 3D illustrates the improved ROSC resulting from the increased CPP illustrated in FIG. 3B.

FIG. 4A is a schematic diagram showing a study design for illustrating the increase in coronary perfusion pressure (CPP) from nasal cooling compared to systemic cooling in a ventricular fibrillation (VF) model of cardiac arrest in pigs.

FIG. 4B illustrates the increase in coronary perfusion pressure (CPP) from nasal cooling compared to systemic cooling in a ventricular fibrillation model (VF) of cardiac arrest in pigs.

FIG. 4C is a table illustrating the improved ROSC resulting from the increased CPP in trans-nasal cooled animals versus IV saline cooled animals.

FIG. 4D is a table illustrating a comparison of the increase in CPP from nasal cooling compared to systemic cooling during chest compressions in a ventricular fibrillation model (VF) of cardiac arrest in pigs.

FIG. 5 illustrates an embodiment of a nasal cooling assembly for use according to the present invention.

FIG. 6 illustrates an embodiment of an oral cooling assembly for use according to the present invention.

FIG. 7A illustrates an embodiment of a nasal catheter for use according to the present invention.

FIG. 7B illustrates an alternative embodiment of a nasal catheter for use according to the present invention.

FIG. 7C illustrates an embodiment of a nasal catheter used to deliver trans-nasal cooling in a pig in the studies illustrated in FIGS. 1-4.

### DETAILED DESCRIPTION OF THE PREFERRED EMBODIMENTS

Resuscitation treatments for patients suffering from cardiac arrest generally include clearing and opening the patient's airway, providing rescue breathing and applying chest compressions to provide blood flow to the victim's heart, brain and other vital organs. When all three treatments are combined, the term cardiopulmonary resuscitation is used. The main factor determining the success of the resuscitation effort following cardiac arrest is coronary perfusion pressure (CPP) during cardiopulmonary resuscitation (CPR). However, CPR alone is typically not sufficient to raise CPP enough for successful resuscitation. When CPP is below 12-14 mm Hg during chest compression, the chances of establishing return of spontaneous circulation (ROSC) are very low. CPR has been shown to increase CPP moderately and epinephrine is also used during resuscitation in an attempt to increase aortic pressure and hence CPP. However, our research has shown that rapid and selective head cooling initiated at the same time as chest compressions or during chest compressions may increase CPP by more than twice as much as CPR alone. This isolated cooling of the nasal or oral cavity and/or cerebral vasculature also has been shown to elevate the CPP by almost twice as much as systemic cooling initiated at the same time as chest compressions or during chest compressions. It is this quick and substantial elevation in CPP that is responsible for the improved rate of ROSC. Moreover, selective head cooling is desirable and preferred over systemic cooling since coagulopathy, poor healing, return of cardiac arrhythmia and cardiac arrest can ensue as a result of systemic cooling.

Specifically, we have determined that cooling the nasal cavity, nasopharynx, oral cavity, oropharynx, retrotonsillar space, mouth, neck, face and/or throat before or during resuscitation from cardiac arrest increases the coronary perfusion pressure (CPP) by augmenting regional sympathetic activity, for example by increasing the sympathetic tone at the aortic root/ascending aorta. This causes increased aortic pressure and therefore increases the coronary perfusion pressure. The anatomy of the brain is such that cooling the nasopharynx and/or oropharyx cools one or more of the cardiac or autonomic control centers, the hypothelamopituitary axis, the midbrain, the pons, the medulla, the autonomic fibers around the brain stem, the cervical sympathetic chain and the thoracic sympathetic chain.

In certain cases, cooling in the nasopharynx and/or oropharynx anesthetizes nerve endings and changes the firing pattern in the cold sensing neurons in the nose, mouth or brain. For example, the typical hemodynamic response to cold exposure is vital organ presentation thus when the hypothalamus receives a message from the oral or nasal cavity nerve endings regarding cooling, the hypothalamus responds by sending a message to increase the CPP. Thus, cooling of the nasopharynx and/or oropharynx can trigger an increase the CPP prior to any noticeable brain cooling.

The increase in CPP and improvement of resuscitation are achieved prior to any systemic or body cooling, and in some embodiments prior to or without any measurable brain cooling. For example, when nasal cooling is initiated at the same time as chest compressions (CPR), CPP increases within seconds, peaks at 2-3 minutes and is still significant 5 minutes into chest compression. The brain temperature, however, does not typically show any measurable decrease until after 15 minutes of nasal cooling. Thus, the beneficial effects of increasing CPP are achieved prior to and without any brain cooling. In some embodiments, the CPP remains elevated for up to 30 minutes, alternatively for at least 10 minutes, alternatively for more than 5 minutes. However, it is the sharp, rapid initial increase in CPP that is responsible for the improved rate of ROSC.

In some embodiments, cooling of the nasal cavity alone is sufficient to augment the regional sympathetic activity and increase the CPP. For example, in some embodiments a reduction in temperature of the nasal cavity by 0.01-20° C. will increase the CPP with out any measurable brain cooling, i.e. the brain temperature is maintained at the patient's baseline brain temperature of between about 35-40° C., preferably about 37° C. In other embodiments, cooling the nasal cavity to increase the CPP may eventually result in a slight reduction in brain temperature without any measurable systemic cooling, i.e. the patient's heart and core body temperature are maintained at a baseline of between about 35-40° C., preferably about 37° C.

In alternative embodiments, an increase in CPP and improvement in ROSC can be achieved by cooling the oral cavity, retrotonsilar space, mouth, neck, throat and/or oropharynx. As discussed above, the oral cavity and oropharynx are in close proximity to the human brain and adjacent to the carotid arteries which supply the brain such that cooling the oral cavity and or oropharynx via direct heat transfer as well as evaporative cooling cools one or more of the cardiac or autonomic control centers, the hypothelamopituitary axis, the midbrain, the pons, the medulla, the autonomic fibers around the brain stem, the cervical sympathetic chain and the thoracic sympathetic chain and augments regional sympathetic activity. For example, in certain embodiments, the temperature in the oral cavity is reduced to between about −10° C. and 25° C., preferably between about −2° C. and 10° C., as measured by a temperature probe in the back of the mouth, to produce an increase in CPP.

When oral cooling is initiated before or during resuscitation, CPP increases within seconds, peaks at 2-5 minutes and remains elevated for up to 30 minutes. In some embodiments, the CPP remains elevated for up to 30 minutes, alternatively for at least 10 minutes, alternatively for more than 5 minutes. However, it is the sharp, rapid initial increase in CPP that is responsible for the improved rate of ROSC. The brain temperature does not typically show any measurable decrease until after 15 minutes of oral cooling. Thus, the beneficial increase in CPP and improvement of resuscitation are achieved prior to any systemic or body cooling, and in some embodiments prior to or without any measurable brain cooling.

Alternatively, the nasal or oral cooling can cause a minimal reduction in brain temperature, for example, by 0.1-0.3 degrees which will result in an increase in CPP. Alternatively, a reduction in brain temperature between 0.01-5° C. will result in an increase in CPP. For example in some embodiments, a 0.01° C., alternatively a 0.1° C., alternatively a 0.5° C., alternatively a 1° C., alternatively a 1.5° C., alternatively a 2° C., alternatively a 3° C., alternatively a 4° C., alternatively a 5° C. or more reduction in brain temperature will result in an increase in CPP.

The location of the nasopharynx and oropharynx in close proximity to the cerebral circulation make it uniquely suited for selective cooling of the brain. The cooling occurs by direct heat transfer through the nasopharynx and/or oropharynx as well as by hematogenous cooling through the carotids as they pass by the oropharynx and through the Circle of Willis, which lies millimeters away from the pharynx. The direct cooling will be obtained through evaporative heat loss of a nebulized liquid in the nasal cavity, oral cavity, and/or throat. Additionally, cooling may occur through convection in the nasal or oral cavity.

A discussed above, cooling the nasal and/or oral cavity produces a rapid and significant increase in CPP compared to chest compressions alone or systemic cooling. The CPP increases within 1 second-30 minutes following cardiac arrest, depending upon when the nasal or oral cooling is initiated and the rate and degree of the cooling. For example, in some embodiments, the CPP increases within 1-15 seconds, alternatively within 1-30 seconds, alternatively within 1-45 seconds, alternatively within 1 second-1 minute, alternatively within 1 second-5 minutes, alternatively within 1 second-15 minutes, alternatively within 1-3 minutes, alternatively within 1-5 minutes, alternatively within 1-15 minutes. This increase in CPP occurs irrespective of the presenting cardiac rhythm, whether ventricular fibrillation (VF) or pulseless electrical activity (PEA). In addition, the degree of nasopharyngeal or oropharyngeal cooling affects the CPP elevation in a dose-dependent manner.

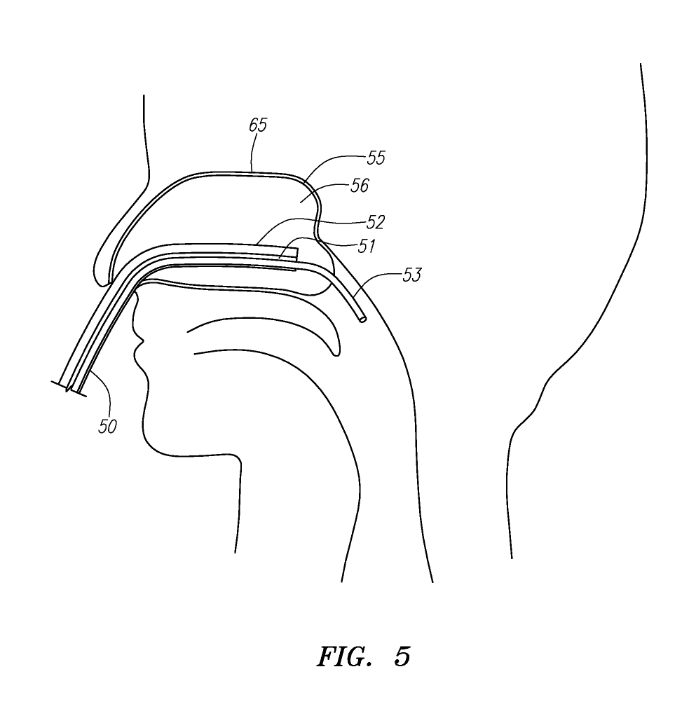

In some embodiments, the coolant such as chilled saline or water can be continuously circulated though the nasal or oral cavity to achieve rapid nasopharyngeal or oropharyngeal cooling. For example, as shown in FIG. 5, a cooling assembly 50 for insertion through a patient's nostril can be used to continuously circulate the coolant into the nasal cavity 65 of a patient. The cooling assembly 50 includes a flexible balloon 55 defining a chamber 56, a first elongate tubular member 51 having a lumen in fluid communication with the chamber 56, and a second elongate tubular member 52 having a lumen in fluid communication with the chamber 56. The cooling assembly 50 may further comprise a third elongate tubular member 53 having a lumen extending from a proximal end to a distal end, wherein the flexible balloon 55 is mounted circumferentially about the third elongate tubular member.

In use, the cooling assembly 50 is inserted into a nasal cavity 65 of a patient through the patient's nostril. A liquid having a temperature between about −20.degree. C. and about 37.degree. C. is infused through the lumen of the first elongate tubular member 51 into the chamber 56 of the flexible balloon 55. The liquid is then withdrawn, suctioned, or drained from the chamber through the lumen of the second tubular member 52. During this process, the chamber 56 of the flexible balloon 55 expands to place the flexible balloon 55 in contact with the nasal cavity 65. The method may further include the step of recirculating the liquid by infusing the liquid through the lumen of the first elongate tubular member and withdrawing the liquid through the lumen of the second elongate tubular member. The liquid may be infused using a pump at a flow rate of between about 5 ml/min and about 5 L/min, alternatively between about 100 ml/min and about 1 L/min, alternatively between about 200 ml/min and about 800 ml/min, alternatively between about 300 ml/min and about 700 ml/min, alternatively between about 400 ml/min and about 600 mL/min, alternatively between about 450 ml/min and about 550 ml/min, alternatively about 500 ml/min. Where the cooling assembly 50 comprises a flexible balloon 55 mounted circumferentially about a third elongate tubular 53 member having a lumen, the third elongate tubular 53 member should be positioned such that the lumen is in fluid communication with the patient's nasopharynx, oropharynx, larynx, and/or esophagus, such that the patient can breathe through the lumen of the third elongate tubular member. Additional embodiments of nasal catheters that can be used to deliver a coolant to a patient's nasal cavity or nasopharynx are further described in co-pending U.S. patent application Ser. No. 11/432,285 filed May 10, 2006 and entitled “Methods and devices for non-invasive cerebral and systemic cooling,” which is hereby incorporated by reference in its entirety.

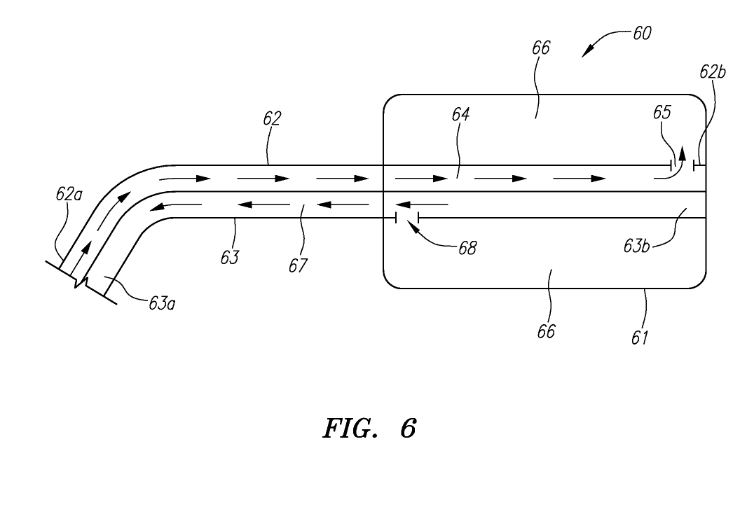

In an alternative embodiment, a cooling assembly 60 for insertion through a patient's mouth can be used to continuously circulate the coolant into the oral cavity of a patient. As shown in FIG. 6, the cooling assembly 60 includes a flexible balloon or pad 61, a first tubular member 62, and a second tubular member 63. The flexible balloon or pad 61 defines a chamber. The first tubular member 62 has a proximal end 62a, a distal end 62b, a lumen therebetween 64, and a port 65 in fluid communication with the lumen 64 of the first tubular member and the chamber 66 of the flexible balloon 61. The second tubular member 63 has a proximal end 63a, a distal end 63b, a lumen therebetween 67, and a port 68 in fluid communication with the lumen of the second tubular member 67 and the chamber 66 of the flexible balloon 61.

In use, the cooling assembly 60 is inserted into a patient's mouth and positioned such that the flexible balloon or pad covers the retromandibular area or the peritonsillar region. A liquid having a temperature between about −20.degree. C. and about 37.degree. C. is infused through the lumen 64 of the first tubular member 62 into the chamber 66 of the flexible balloon 61. The liquid is then withdrawn, drained, or suctioned from the chamber through the lumen 67 of the second tubular member 63. During this process, the chamber 66 of the flexible balloon 61 or pad expands to place the flexible balloon 61 in contact with the adjacent anatomy, i.e., the retromandibular area or the peritonsillar region. The method may further include the step of recirculating the liquid by infusing the liquid through the lumen of the first tubular member and withdrawing the liquid through the lumen of the second tubular member. The liquid may be infused using a pump at a flow rate of between about 5 ml/min and about 5 L/min, alternatively between about 100 ml/min and about 400 ml/min, alternatively between about 150 ml/min and about 200 ml/min. Additional embodiments of cooling assemblies that can be used to deliver a coolant to a patient's oral cavity or oropharynx are further described in co-pending U.S. patent application Ser. No. 12/101,933, filed on Apr. 11, 2008 and entitled “Methods and Devices for Non-invasive Cerebral and Systemic Cooling,” which is hereby incorporated by reference in its entirety.

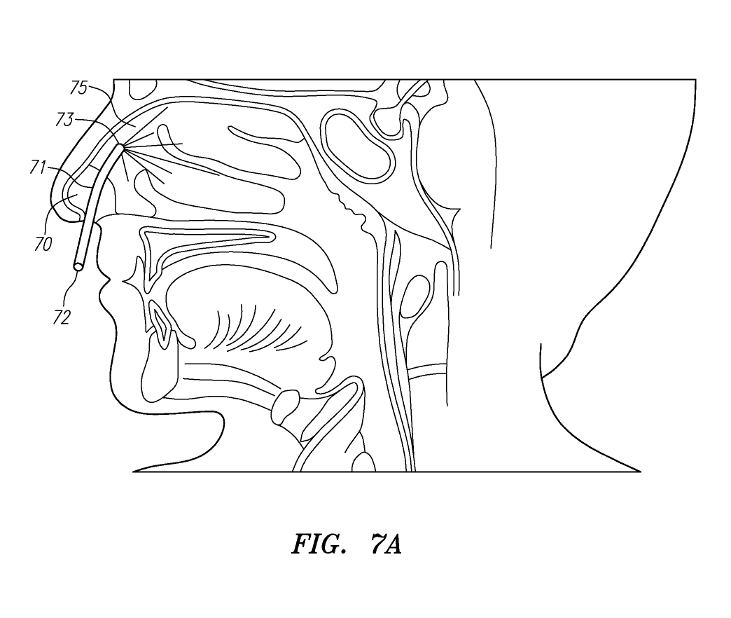

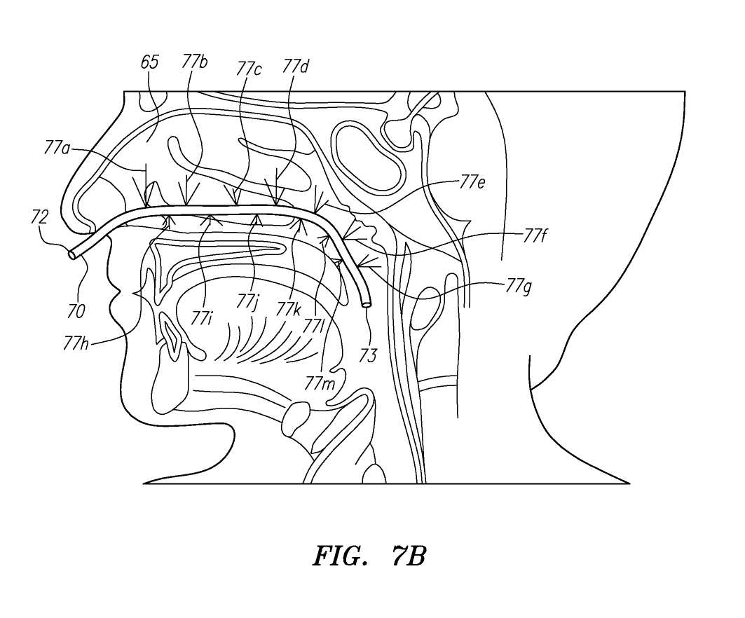

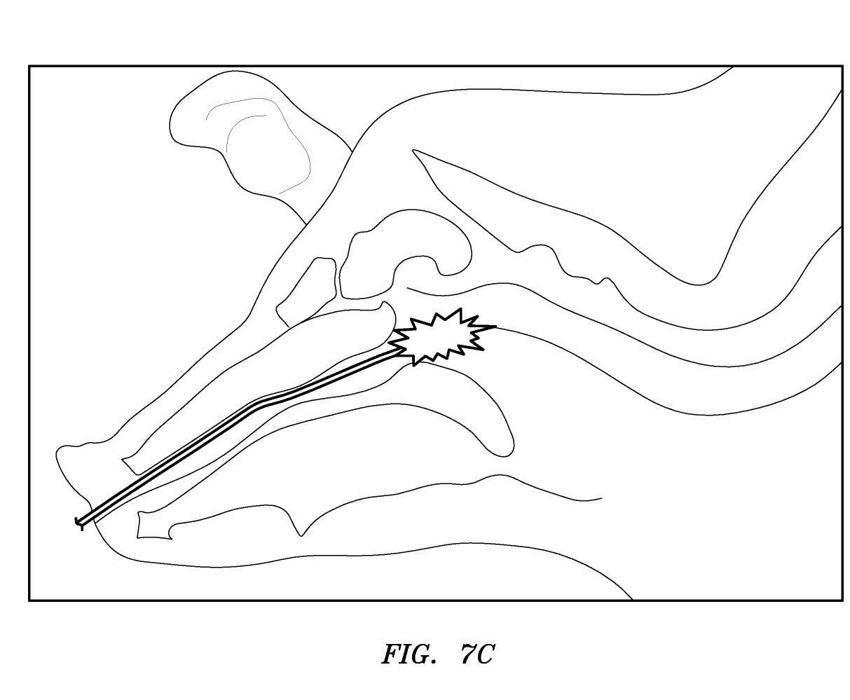

Alternatively, intermittent cooling can be used with a more volatile, colder coolant such as a perfluorocarbon or a hydrocarbon to allow the coolant time to remove heat without freezing the surrounding tissue. For example, in some embodiments, the cooling can be provided according to a duty cycle of between 5-80%. In some embodiments, the coolant can be circulated with a gas to enhance evaporation of the coolant. For example, the coolant can be delivered onto the surface of the patient's nasal or oral cavity as a pulsatile spray. For example, as shown in FIG. 7A, in some embodiments, a nasal catheter 70 having an elongate member 71 can be inserted into a nasal cavity 75 of a patient through the patient's nostril to deliver a spray of liquid to the nasal cavity 75 of a patient. The nasal catheter 70 may be placed in the nares of the patient's nose and may be angled to direct the spray outlet at the desired anatomic location, for example the nasopharynx. The elongate member 71 may have a proximal end 72, a distal end 73, a first lumen extending therebetween, and one or more of ports on the distal end 73 in fluid communication with the first lumen. A perfluorocarbon spray is then delivered onto a surface of the patient's nasal cavity through the plurality of ports in the distal end. In addition, the distal end 73 of the nasal catheter and may be designed to cause the spray to spread in a pattern which will allow the gas and liquid mixture to contact as much of the desired tissue as possible. The evaporation of the perfluorocarbon from the nasal cavity results in rapid cooling of the nasal cavity. In some embodiments, the nasal catheter may have a second elongate tubular member for placement in the patient's other nostril to maximize the rate of coolant delivery.

Alternatively, as shown in FIG. 7B, a nasal catheter 70 comprising an elongate tubular member 71 and a plurality of ports 77a-m extending along a length of the elongate tubular member 71 may be inserted in to the patient's nasal cavity 75 and positioned such that the plurality of ports 77a-m are positioned to deliver the cooled liquid and/or gas mixture on the surface of the nasal cavity, nasopharynx, and or oropharaynx. In some embodiments, the nasal catheter may be used to deliver a perfluorocarbon and a gas, such as oxygen. The gas may enhance the evaporation of the perfluorocarbon further increasing the cooling effect. Additional embodiments of nasal catheters that can be used to deliver a intermittent cooling to a patient's nasal cavity or nasopharynx are further described in co-pending U.S. patent application Ser. No. 11/432,285, filed May 10, 2006 and entitled “Methods and Devices for Non-invasive Cerebral and Systemic Cooling,” which is hereby incorporated by reference in its entirety.

### EXAMPLES

The following examples are offered to illustrate but not limit the claimed invention.

### Example 1

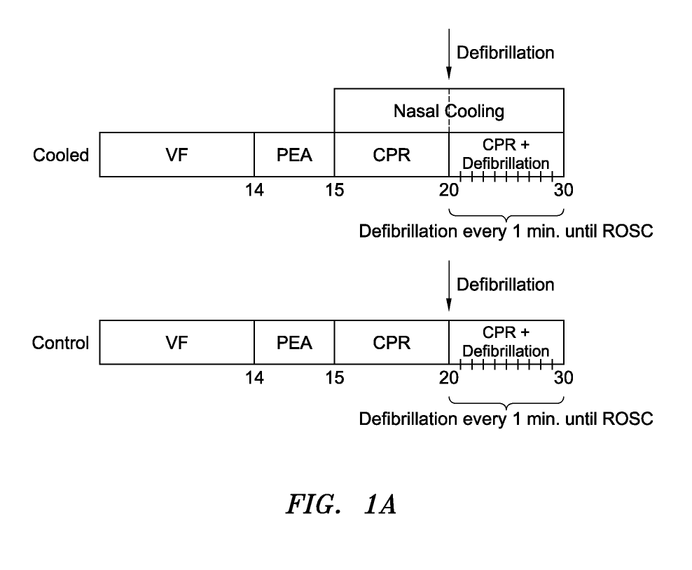

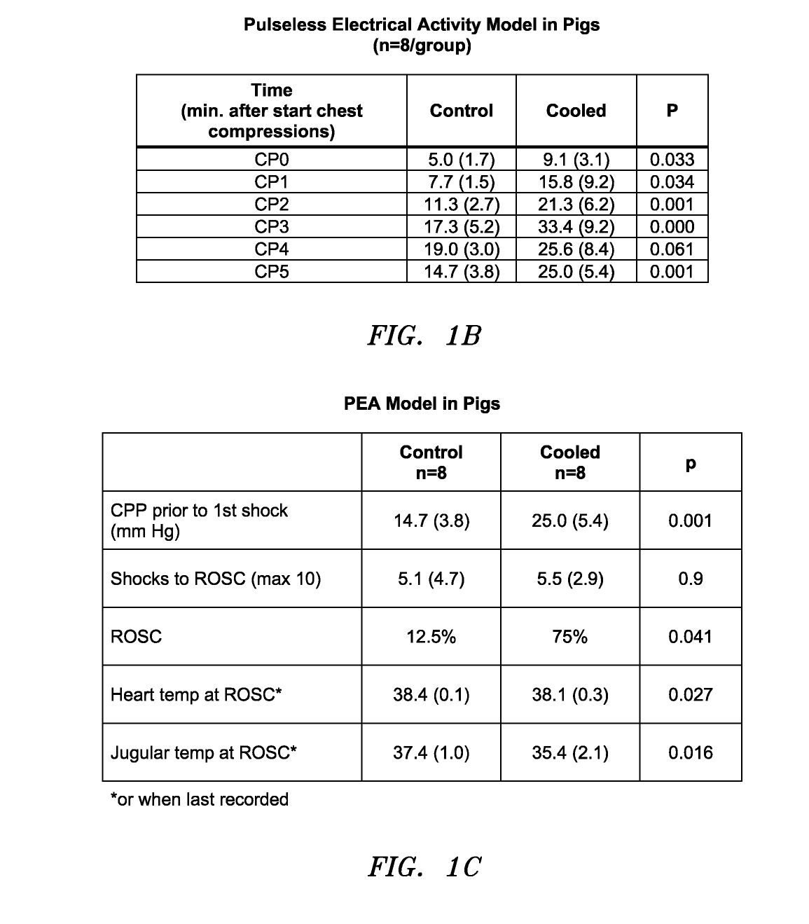

The following study illustrates the effect of selective head cooling on coronary perfusion pressure (CPP) after pulseless electrical activity (PEA). As shown in FIG. 1A, in this study ventricular fibrillation (VF) was electrically induced in 16 male domestic pigs weighing 40±3 kg. After 14 minutes of untreated VF, PEA was induced by one or more electrical shocks. After one minute of PEA, CPR including chest compression and ventilation with oxygen was begun. In 8 animals, trans-nasal head cooling was begun coincident with CPR. As shown in FIG. 7C, a trans-nasal catheter, a modified Rhinochill™ device (BeneChill, San Diego, Calif.) was used to deliver the coolant. The coolant, perfluorohexane (PFH), was delivered at a rate of 0.6-0.8 mL/min/kg body weight and was continued for a minimum of 15 minutes. If the animals achieved return of spontaneous circulation (ROSC) within the 15 minute period, the cooling was continued for 4 hours. The remaining 8 randomized controls were identically treated except for the head cooling. CPR was continued for both the cooled and control animals for five minutes prior to the first attempted defibrillation. CPR was then continued for 1 minute intervals in between defibrillation attempts until ROSC or for a total of 15 minutes.

FIG. 1B shows the increase in CPP for the cooled animals compared to non-cooled animals during CPR in the PEA cardiac arrest model. CP stands for chest compression (CPR) and the number represents duration of CPR in minutes (CP1=chest compression 1 minute). The increase in CPP for the cooled animals is immediate, maximal at 3 minutes and significant at 5 minutes. The CPP increased in both groups because of chest compressions. However, the initial increase was quicker in the cooled group and the overall increase was twice as much in the cooled group compared to the non-cooled group. As shown in FIG. 1C, the increased CPP in the cooled animals correlates with an improved rate of return of spontaneous circulation (ROSC). In the cooled group the average CPP at the first shock was 25.0±5.4 and six of the eight animals were resuscitated whereas only in the control group where the average CPP at first shock was 14.7±5.4 only one animal was resuscitated. Moreover, the number of shocks required to achieve ROSC was less in the cooled group.

In this study, trans-nasal cooling was initialed at the same time as the CPR and maintained for fifteen minutes. As illustrated by FIG. 1B, one minute of cooling is typically sufficient to increase the CPP and five minutes of cooling ensures that the CPP remains elevated for at least 10 minutes, alternatively 15 minutes, alternatively 30 minutes. Accordingly, cooling is usually performed for less than 20 minutes, alternatively between about 10-20 minutes, alternatively no more than 5 minutes.

### Example 2

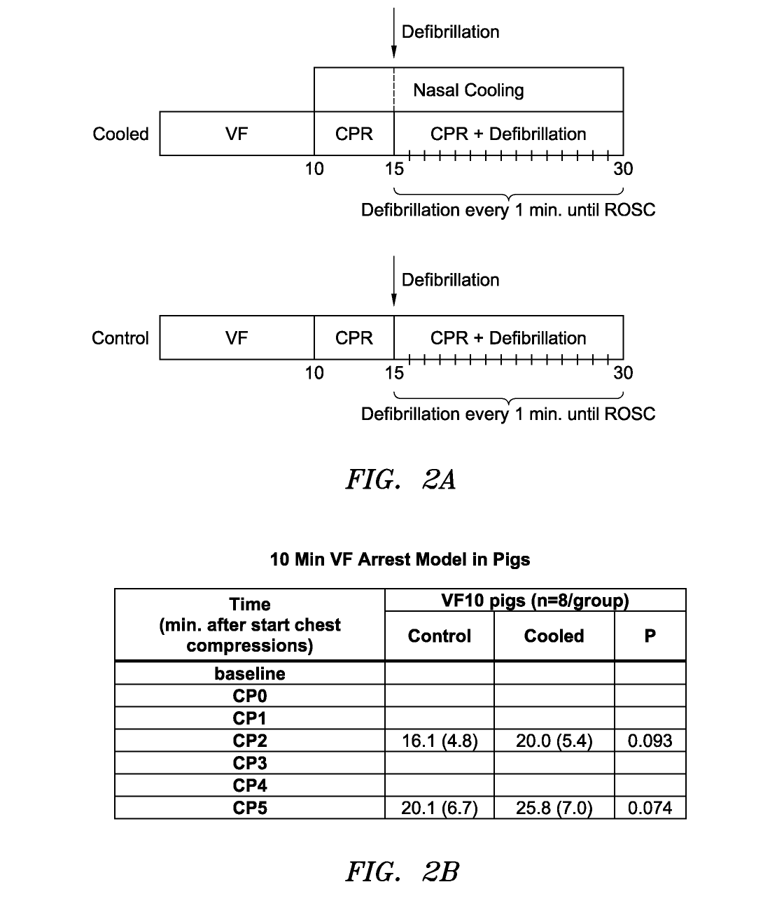

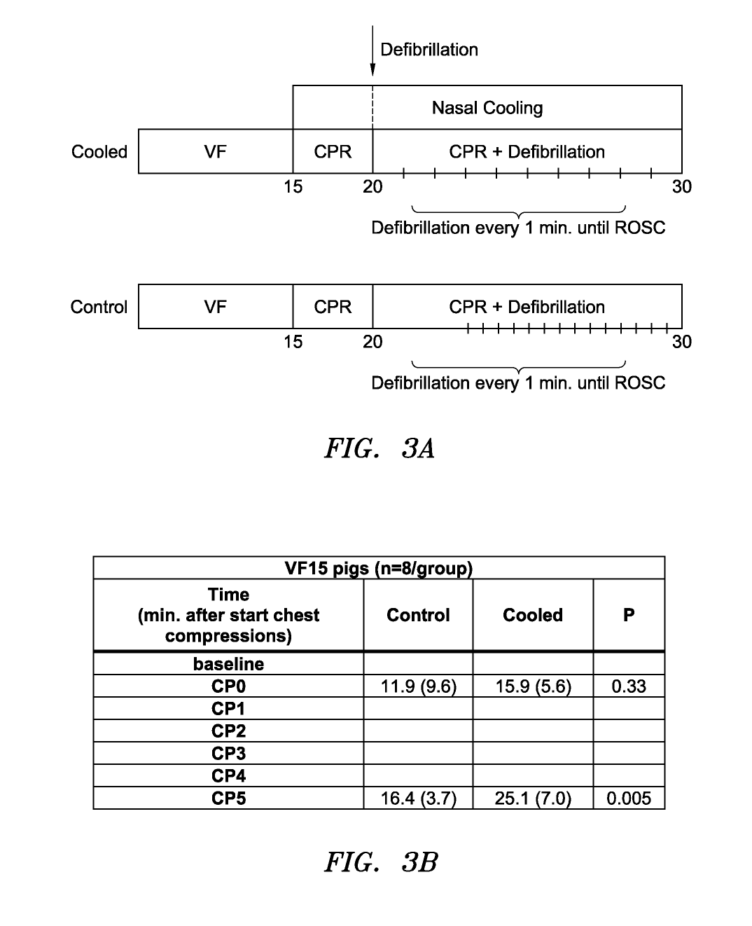

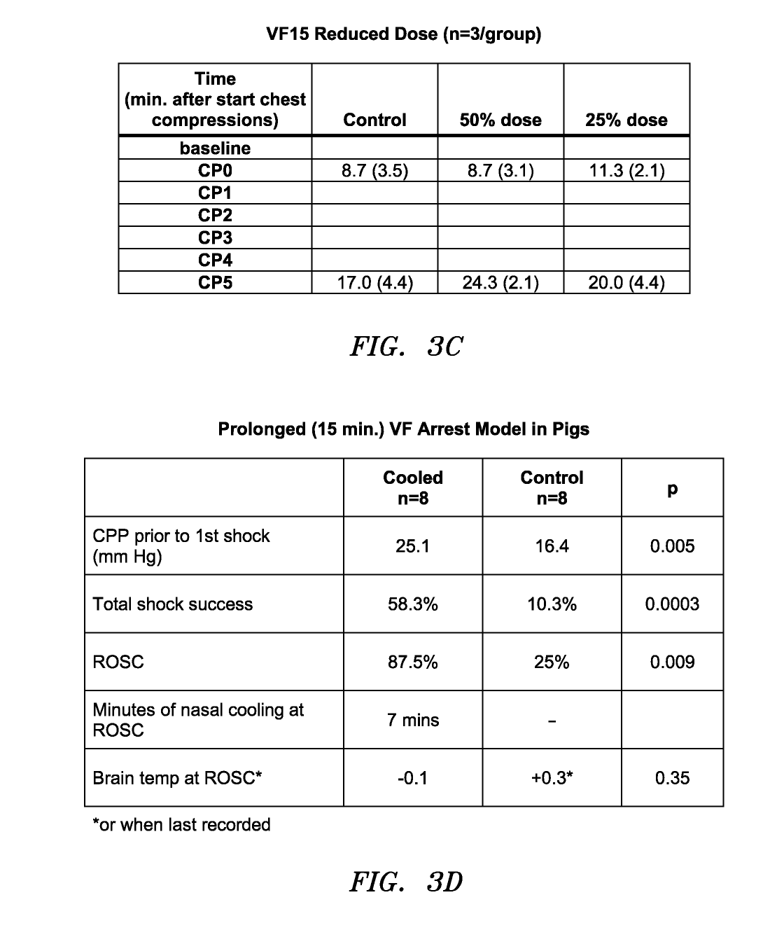

FIGS. 2-3D show the effects of selective head cooling on increasing coronary perfusion pressure (CPP) resulting in an improved ROSC in a ventricular fibrillation (VF) model of cardiac arrest in pigs. In FIG. 2A, VF was electrically induced in 16 male domestic pigs left untreated for 10 minutes. After 10 minutes of untreated VF, CPR including chest compression and ventilation with oxygen was begun. In 8 animals, selective head cooling was begun coincident with CPR. As shown in FIG. 7C, a trans-nasal catheter, a modified Rhinochill™ device (BeneChill, San Diego, Calif.) was used to deliver the coolant. The coolant, perfluorohexane (PFH), was delivered at a rate of 0.6-0.8 mL/min/kg body weight and was continued for a minimum of 15 minutes. If the animals achieved return of spontaneous circulation (ROSC) within the 15 minute period, the cooling was continued for 4 hours. The remaining 8 randomized controls were identically treated except for the head cooling. CPR was continued for both the cooled and control animals for five minutes prior to the first attempted defibrillation. CPR was then continued for 1 minute intervals in between defibrillation attempts until ROSC or for a total of 15 minutes.

As shown in FIG. 2B, the increase in CPP for the cooled animals is significant at 2 minutes and remains elevated at 5 minutes. The CPP increased in both groups because of chest compression; however the increase was greater in the cooled group.

FIG. 3A illustrates a similar study on the effects nasal cooling on CPP after prolonged VF. In this study, VF was electrically induced in 16 male domestic pigs weighing 40±2 kg and left untreated for 15 minutes. After 15 minutes of untreated VF, the animals were randomized to either cooled or control groups and CPR including chest compression and ventilation with oxygen was begun. In the cooled group, selective head cooling was begun coincident with CPR. The animals were cooled via a nasal catheter inserted into the nostrils of the pigs which produced evaporative cooling in the nasopharynx. As shown in FIG. 7C, a trans-nasal catheter, a modified Rhinochill™ device (BeneChill, San Diego, Calif.) was used to deliver the coolant. The coolant, perfluorohexane (PFH), was delivered at a rate of 0.6-0.8 ml/min/kg body weight and was continued for a minimum of 15 minutes. If the animals achieved return of spontaneous circulation (ROSC) within the 15 minute period, the cooling was continued for 4 hours. The remaining 8 randomized controls were identically treated except for the head cooling. CPR was continued for both the cooled and control animals for five minutes prior to the first attempted defibrillation. If ROSC was not restored, CPR was resumed for 1 minute prior to another defibrillation attempt until successful ROSC or for a total of 15 minutes.

As shown in FIG. 3B, the CPP increased in both the control and cooled animals; however the increase in the cooled animals was almost twice as much. FIG. 3D illustrates that the increased CPP in the cooled animals further correlates with an improved rate of return of spontaneous circulation (ROSC). In the cooled group seven of the eight animals were resuscitated whereas only two animals in the control group were resuscitated. The average CPP at the time of the first shock was 25.1 mm Hg compared to 16.4 mm Hg for the control group. Moreover, at ROSC which was attained after an average of seven minutes of trans-nasal cooling, the cooled animals exhibited no significant reduction in brain temperature. Thus, the increase in CPP responsible for the improved ROSC was achieved as a result of the nasal cooling alone and without any statistically significant reduction in brain temperature.

As shown in FIGS. 3A-D, even after 15 minutes of arrest, the CPP increases within seconds or minutes of initiating cooling. The increase in CPP for the cooled animals is evident at 1 minute and significant at 5 minutes. Again, the CPP increased in both the control and cooled animals; however the increase in the cooled animals was almost twice as much. Thus, CPP increases with nasopharyngeal and/or oral cooling irrespective of the duration of the cardiac arrest.

FIGS. 3B-C illustrate the dose-dependent effect of cooling on the CPP. The study was performed as described above with reference to FIG. 3A, wherein the animals were subject to 15 minutes untreated VF and then selective head cooling was begun coincident with CPR. The animals were cooled via a nasal catheter inserted into the nostrils of the pigs. CPR was continued for five minutes prior to the first attempted defibrillation. If ROSC was not restored, CPR was resumed for 1 minute prior to another defibrillation attempt until successful ROSC or for a total of 15 minutes. The coolant, perfluorohexane (PFH), was delivered at a rate of 0.8 mL/kg/min in the animals receiving the maximum dose, 0.4 mL/kg/min in the animals receiving the moderate dose and 0.2 mL/kg/min in the animals receiving the low dose. As shown in FIG. 3C, at a low dose (25% coolant flow rate) the effect on CPP is less marked than at moderate dose (50% coolant flow rate), and maximal effect is seen at highest coolant flow rates (“cooled”). However, in all three flow rates, the CPP increased significantly compared to the non-cooled (“control”) animals. Thus, the coolant, such as chilled saline, water, water containing a nanotech particle, a PFC, a hydrocarbon, or an ice slurry could be provided at a flow rate of between about 1 ml/min-500 ml/min in order to achieve a beneficial increase in CPP and improved rate of ROSC. Depending on the coolant selected, the coolant can be circulated through the anatomic location continuously or intermittently.

### Example 3

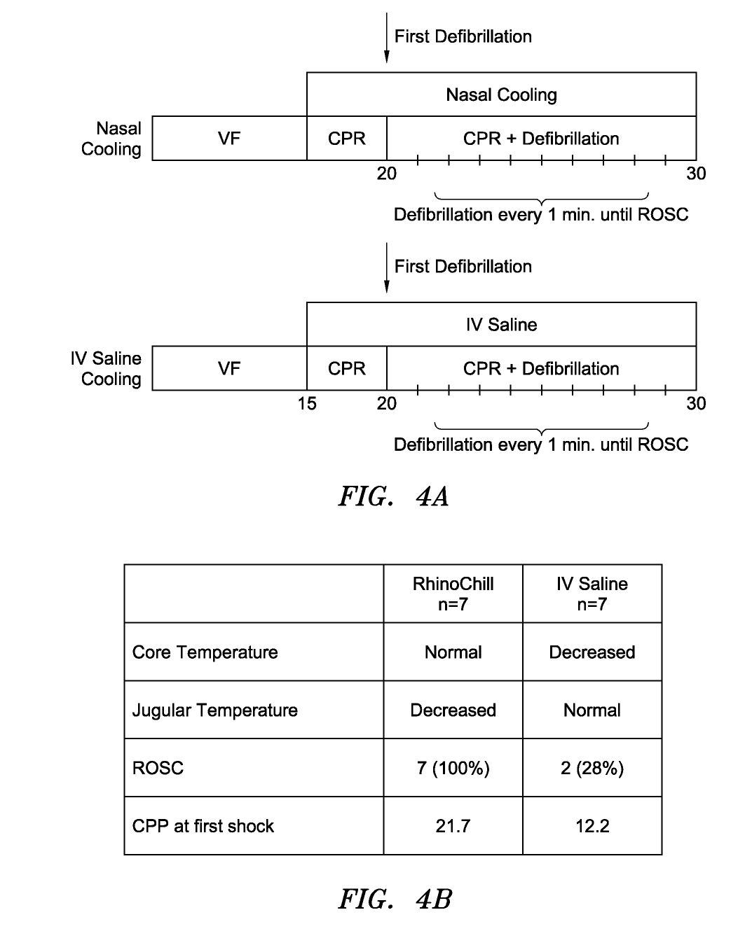

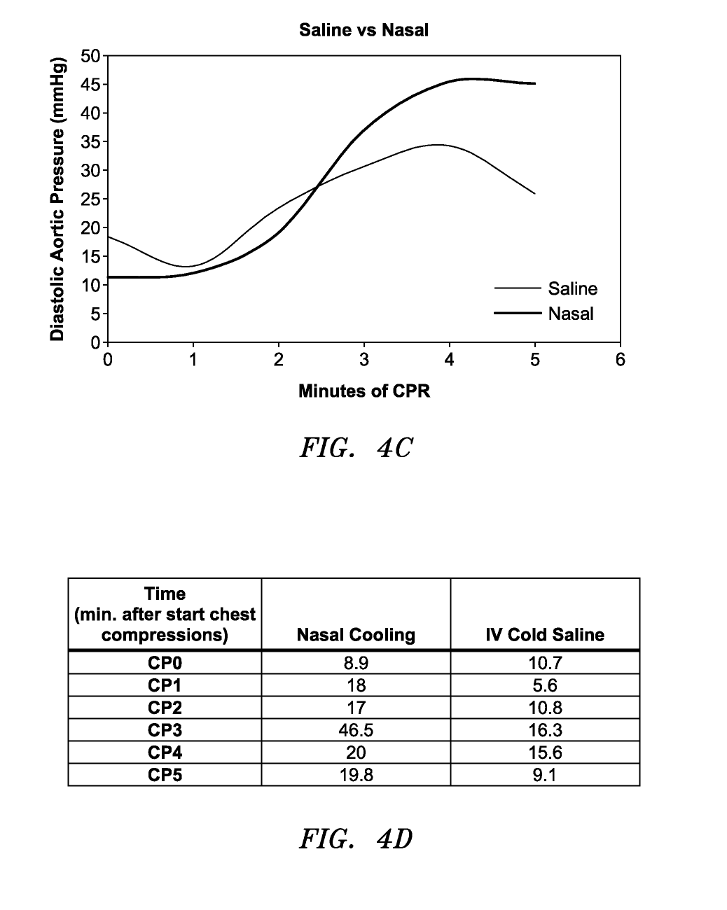

FIGS. 4A-D describe a study which illustrates that the beneficial effect on CPP is specific to this method of selective nasal or oral cooling and is unrelated to systemic hypothermia. As shown in FIG. 4A, in this study, ventricular fibrillation (VF) was electrically induced in 14 male domestic pigs weighing 37±3 kg and left untreated for 15 minutes. After 15 minutes of untreated VF, CPR including chest compression and ventilation with oxygen was begun.

In 7 animals, trans-nasal cooling was begun coincident with CPR using a Rhinochill nasal catheter (BeneChill, San Diego, Calif.). The coolant delivery was scaled to the animal size to deliver 0.6-0.8 mL/min/kg body weight. It was continued for a minimum of 15 minutes; if the animals achieved return of spontaneous circulation (ROSC) within this 15 minute period, then cooling was continued for four hours. In the remaining 7 animals, an IV saline drip was begun coincident with CPR. Here, a 4° C. saline was introduced intravenously at the rate of 30 mL/kg body weight for 30 minutes. CPR, including mechanical chest compressions and ventilation, was continued for 5 minutes prior to the first attempted defibrillation. CPR was then continued for one minute intervals in between defibrillation attempts until ROSC was achieved or for a total of 15 minutes.

As shown in FIG. 4C, the increase in CPP for the trans-nasally cooled animals is significant at 2 minutes and remains elevated at 5 minutes. The CPP increased in both trans-nasally cooled animals and IV cooled animals because of chest compression, however the increase was greater in the trans-nasally cooled group. Furthermore, in the trans-nasally cooled group, the CPP remained elevated at five minutes. Conversely, in the IV saline cooled group, the initial increase in CPP diminishes by five minutes as a result of the increase in right atrial pressure from the volume overload of the cold saline infusion. As shown in FIG. 4B, the increased CPP resulting from trans-nasal cooling is significantly higher that the CPP resulting from systemic cooling via IV saline. Moreover, the increased CPP correlates with an improved rate of return of spontaneous circulation (ROSC). Specifically, it is the rapid, sharp increase in CPP that is responsible for the improved rate of ROSC. As shown in FIG. 4C, the increased CPP in the cooled animals correlates with an improved rate of return of spontaneous circulation (ROSC). In the nasal cooled group the average CPP at the first shock was 21.7 mm Hg and seven out of seven animals were resuscitated whereas only in the IV saline cooled group where the average CPP at first shock was 12.2 mm Hg only two out of seven animals were resuscitated.

As shown in FIG. 4C, CPP increase, attributable to the increase in aortic root diastolic pressure, starts within 1-2 minutes of initiating cooling, peaks around 5 minutes and continues to remain elevated for 15-30 minutes. CPP also rises in the IV saline group as a result of the chest compressions, but this is not nearly as significant as in the trans-nasally cooled group and is not sustained. Rather, the increase in aortic root pressure which accounts for the increased CPP in the trans-nasally cooled animals is caused by regional sympathetic simulation which is likely a result of cooling one or more of the cardiac or autonomic control centers, the hypothelamopituitary axis, the midbrain, the pons, the medulla, the autonomic fibers around the brain stem, the cervical sympathetic chain and the thoracic sympathetic chain. As shown in another study illustrated above in FIGS. 3A-D, this increase in CPP from trans-nasal cooling is rapid and selective, resulting before any measurable decrease in brain temperature. Specifically, as discussed above, the increase in CPP starts within 1-2 minutes of initiating cooling whereas, as shown in FIG. 3D, the brain temperature even after 7 minutes of cooling has undergone no measurable decrease.

In a similar study, shown in FIG. 4D, when systemic hypothermia is induced using 2° C. saline introduced intravenously, CPP does not increase and may in fact decrease. Here, with localized nasopharyngeal cooling, the CPP increased significantly within the first minute to an average of 18 mm of Hg, showed maximal increase of an average of 46.5 mm of Hg at three minutes and remained significant at an average of 19.8 mm of Hg at 5 minutes. Conversely, the animals treated with systemic cooling via the IV saline, did not show a rapid significant increase in CPP and in fact began to show a decrease in CPP by five minutes as a result of the increase in the right atrial pressure from the volume overload of the cold saline infusion. This gives nasal cooling an advantage over IV saline as a myocardial protectant.

In addition, increasing CPP may be valuable in settings other than cardiac arrest. For example, it may be beneficial wherever CPP is low, such as myocardial infarction, cardiac failure, cardiac surgery, non-surgical coronary vessel procedures and even neurologic injuries associated with low CPP.

While embodiments of the present invention have been shown and described, various modifications may be made without departing from the scope of the present invention. The invention, therefore, should not be limited, except to the following claims, and their equivalents.

What is claimed is:

1. A method for increasing coronary perfusion pressure to improve success of return of spontaneous circulation (ROSC) after cardiac arrest, comprising the steps of:
selecting a patient suffering from cardiac arrest;
cooling an anatomic location selected from the group consisting of, nasopharynx, nasal cavity, oropharynx, retrotonsillar space, mouth, neck and throat;
increasing the coronary perfusion pressure; and
administering to the patient at least one of chest compressions, defibrillation and vasoconstrictor.

2. The method of claim 1, wherein the cooling increases the coronary perfusion pressure within 1-5 minutes.

3. The method of claim 1, wherein the cooling increases the coronary perfusion pressure within 1-3 minutes.

4. The method of claim 5, wherein the cooling increases the coronary perfusion pressure within 1-30 seconds.

5. The method of claim 5, wherein the cooling increases the coronary perfusion pressure within 1-15 seconds.

6. The method of claim 1 wherein the coronary perfusion pressure remains elevated for more than 3 minutes.

7. The method of claim 6, wherein the coronary perfusion pressure continues to increase for more that 3 minutes.

8. The method of claim 1 wherein the coronary perfusion pressure remains elevated for more than 5 minutes.

9. The method of claim 8, wherein the coronary perfusion pressure remains elevated for at least 10 minutes.

10. The method of claim 1, wherein the coronary perfusion pressure is increased without reducing the brain temperature.

11. The method of claim 1, wherein coronary perfusion pressure is increased while maintaining the patient's baseline brain temperature at 37° C.

12. The method of claim 1, wherein coronary perfusion pressure is increased while maintaining the patient's brain temperature between 35-40° C.

13. The method of claim 1, wherein the temperature in the brain is reduced by 0.01-5° C.

14. The method of claim 1, wherein the temperature in the nasal cavity is reduced by 0.01-20° C.

15. The method of claim 14 wherein the cooling step reduces the temperature in the nasal cavity by 0.01-20° C. while maintaining the patient's baseline brain temperature at 37° C.

16. The method of claim 1, wherein the cooling step reduces the temperature in the mouth to between −10° C. and 30° C.

17. The method of claims 16, wherein the cooling step reduces the temperature in the mouth to between −2° C. and 10° C.

18. The method of claim 1, wherein the cooling step comprises cooling the anatomic location for less than 20 minutes.

19. The method of claim 1, wherein the cooling step comprises cooling the anatomic location for 10-20 minutes.

20. The method of claim 1, wherein the cooling step comprises cooling the anatomic location for no more that 5 minutes.

21. The method of claim 1, wherein the cooling step comprises circulating a cold liquid through the anatomic location.

22. The method of claim 21, wherein the cold liquid is selected from the group consisting of chilled saline, water, water containing a nanotech particle, a PFC, a hydrocarbon, or an ice slurry.

23. The method of claim 22, wherein the cold liquid is circulated at a flow rate of about 1 ml/min-500 ml/min.

24. The method of claim 21, wherein the cold liquid is circulated intermittently.

25. The method of claim 24, wherein the intermittent circulation has a duty cycle of between 5-80%.

26. The method of claim 24, wherein the intermittent circulation comprises using a pulsatile spray to deliver the cold liquid.

27. The method of claim 1, wherein the cooling step comprises circulating a cold gas through the anatomic location.

28. The method of claim 1, wherein the cooling step comprises circulating a cold liquid and a gas mixture through the anatomic location.

29. The method of claim 1, wherein the cooling step further comprises cooling one or more of the cardiac control centers in the brain.

30. The method of claim 29, wherein cooling one or more of the cardiac control centers changes the firing pattern in cold sensing neurons in the mouth or nose or in the underside frontal lobe or preoptic region of the brain or brain stem.

31. The method of claims 1, wherein cooling the nasopharynx further comprises cooling one or more of the hypothelamopituitary axis, the midbrain, the pons, the medulla, the autonomic fibers around the brain stem, the cervical sympathetic chain and the thoracic sympathetic chain.

32. The method of claim 1, wherein the cooling increases the coronary perfusion pressure by 50% within the first minute without reducing the brain temperature.
